(19) United States
(12) Patent Application Publication
(10) Pub. No.: US 2006/0183974 A1
(43) Pub. Date: Aug. 17, 2006

(54) Endoscope With Improved Maneuverability

(75) Inventors: Avi LEVY, Herzlyia (IL); Shai BRENNER, Haifa (IL); Dan OZ, Yehuda (IL)

Correspondence Address: BRUCE E. LILLING;LILLING & LILLING P.C., P.O. BOX 560, GOLDEN BRIDGE, NY, 10526, US

(73) Assignee: SIGHTLINE TECHNOLOGIES LTD., Haifia (IL)

(21) Appl. No.: 11/275,965

(22) Filed: Feb. 7, 2006

Related U.S. Application Data
(60) Provisional application No. 60/652,586, filed on Feb. 14, 2005.

(51) Int. Cl. A61B 1/12; A61B 1/00

(52) U.S. Cl. 600/116; 600/114; 600/156

(57) ABSTRACT

Apparatus and method for endoscopic examination of a tubular conduit are described. The apparatus is provided with an insertion tube fitted at a distal end thereof with an optical head for visualization of the conduit's interior. The insertion tube is coupled with a major inflatable sleeve, which upon inflation is capable of propelling the endoscope within the conduit. The insertion tube is also coupled with the at least one auxiliary inflatable sleeve, which upon inflation thereof provides additional propelling force for advancement of the insertion tube within the conduit. By inflating the major sleeve and the at least one auxiliary sleeve, it is possible to distribute the propulsion force along the insertion tube.

### FIELD OF THE INVENTION

The present invention relates generally to the field of endoscopy and specifically to endoscopes used for colonoscopic procedures, during which a flexible endoscope is inserted into the rectum and colon for examination of the colon interior for abnormalities. More particularly, the present invention refers to such an endoscope, which is provided with more than one disposable sleeve covering the insertion tube and enabling, upon inflation, propulsion of the endoscope.

It should be kept in mind that the present invention is not limited strictly to endoscopes for use in the medical field. It also can be implemented in endoscopes used for non medical applications, e.g. in borescopes suitable for various engineering applications where it is required to investigate the interior of a tubular conduit bent in a complex shape or to pass a cable or any other pliable elongate object through such a tubular conduit.

### BACKGROUND OF THE INVENTION

It is well known that advancement of an endoscope during a colonoscopic procedure is a difficult task, since the probe of the endoscope should pass many sections of the body passage, including rectum, sigmoid colon, descending colon, transverse colon, ascending colon, and then the cecum. Altogether these sections define a very complicated, bent labyrinth, consisting of several turns, which the probe should pass. There are known endoscopes employing inflatable flexible sleeves to assist the colonoscopist in advancing the endoscope through a body passage.

Voloshin (U.S. Pat. No. 6,485,409) discloses an endoscope, which comprises an endoscopic probe, a bending section for directing the probe within the colon (steering unit), an insertion tube and a flexible covering sleeve or a sheath, which is coupled proximally to the probe. The bending section of the colonoscope is located behind the probe. The sleeve is attached to the endoscope in such a manner that its folded section is retained between a cup and an internal spindle, which are located between the insertion tube and the steering unit. When inflated, the folded section unfolds over a flange of the internal spindle and an inner portion of the sleeve is pulled behind the steering unit in a distal direction. Since the sleeve covers merely the insertion tube and neither the steering unit nor the probe, they will be contaminated during the colonoscopic procedure and therefore the entire endoscope should be disinfected before reuse.

Eizenfeld (International application PCT/IL03/00661; Publication WO 2004/016299) describes an endoscope employing a flexible inflatable sleeve, which before inflation is retained within a dispenser. To protect the endoscope from contamination the sleeve is put thereon in such a manner that it covers the insertion tube, bending section and the probe. Advancing the endoscope in this manner causes the sleeve to feed out gradually, so that it covers that part of the endoscope which is inside the body passage, thus protecting it from contamination. The sleeve may be inflated in order to reduce friction between the sleeve and the endoscope as the endoscope advances. Unfortunately this measure reduces to some extent the maneuverability of the bending section, since inflation of the sleeve imparts some rigidity to the forward extremity of the probe. Insufficient maneuverability renders advancement of the endoscope within the colon more difficult and accordingly operation of the endoscope is less convenient. Baror (International patent application PCT/IL01/00778; Publication WO 02/19886) describes an endoscopic apparatus comprising a probe with an anterior component and a posterior component and a flexible dual-sleeved tube which enclosure is inflated in order to propel the anterior component within a lumen. Construction of this apparatus is complicated and does not allow independent inflation of the sleeves.

Bar-or (International patent application PCT/IL05/000425; Publication WO 2005/110204) discloses an apparatus for endoscopic examination of a body passage. This apparatus is provided with a flexible endoscope and with an inflatable disposable sleeve detachably connected to the endoscope. Upon inflation the sleeve assists in propelling the endoscope within the body passage. The endoscope is provided with a proximal end and with a distal end, wherein the distal end comprises an insertion tube, a bending section and a probe fitted with an optical head. The sleeve comprises a rear folded portion, which unfolds upon inflation and a frontal, non-inflatable portion, which covers the bending section and the probe. The sleeve is coupled to the endoscope by an anchoring and sealing mechanism, which allows attachment of the endoscope to the sleeve at the beginning of the endoscopic procedure and its detachment at the end of the endoscopic procedure, wherein said anchoring and sealing mechanism seals the frontal portion of the sleeve from inflation.

The reaction force, arising due to the inflation of the flexible sleeve, advances the endoscope along the body passage. This force, which is applied at the distal end, and the pushing force applied by an operator at the proximal end are the driving forces available in the prior art endoscopic devices. Unfortunately none of the above devices provides an adequate solution of the maneuverability problem arising during advancement of the endoscope through the interior of a complicated body passage. One of the reasons for this is associated with the fact that none of them provides a possibility of applying a driving force to an intermediate section of the endoscope as might be required when it entangles.

Furthermore body tissues along the body passage are not homogeneous and therefore various locations of the body passage have various resistances, which renders advancement of the endoscope even more difficult.

### SUMMARY OF THE INVENTION

The object of the present invention is to provide an endoscope, which has improved maneuverability during advancement through turns and loops of a body passage.

Still further object of the invention is to provide a new and improved endoscope, which is less prone to entanglement within the body passage. In accordance with the present invention the above and other objects are achieved by providing the endoscope with several inflatable disposable sleeves, which can be selectively inflateable to provide additional driving force applied to those portions of the endscope, which are located between the distal and the proximal end and thus assisting advancement of the endoscope through those locations of the body passage, which have complicated configurations and different material structures.

It should be kept in mind that the present invention still provides full protection of the endoscope from contamination, since inflatable, disposable sleeves cover the insertion tube, the bending section and the probe.

For a better understanding of the present invention as well of its benefits and advantages, reference will now be made to the following description of its embodiments taken in combination with the accompanying drawings.

### BRIEF DESCRIPTION OF THE DRAWINGS

FIG. 1 is a general view of a prior art endoscopic apparatus and its main components.

FIG. 2a depicts an endoscopic apparatus of the present invention within a body passage.

FIG. 2b depicts an endoscopic apparatus of the present invention outside the body passage.

FIG. 3 is a partial representation of an embodiment of the attachment mechanism suitable for engagement propelling sleeves.

FIG. 4 shows schematically propelling sleeves when they all are engaged and inflated.

FIG. 5 shows channels for automatic, sequential supplying of inflating medium.

FIG. 6 shows channels for independent supplying of inflating medium.

FIG. 7 shows schematically an embodiment of the attachment and inflating mechanism in a situation when one of the sleeves is not engaged yet.

FIG. 8 shows schematically the mechanism of FIG. 7 in a situation when the sleeve is engaged and inflated.

### DETAILED DESCRIPTION OF THE INVENTION

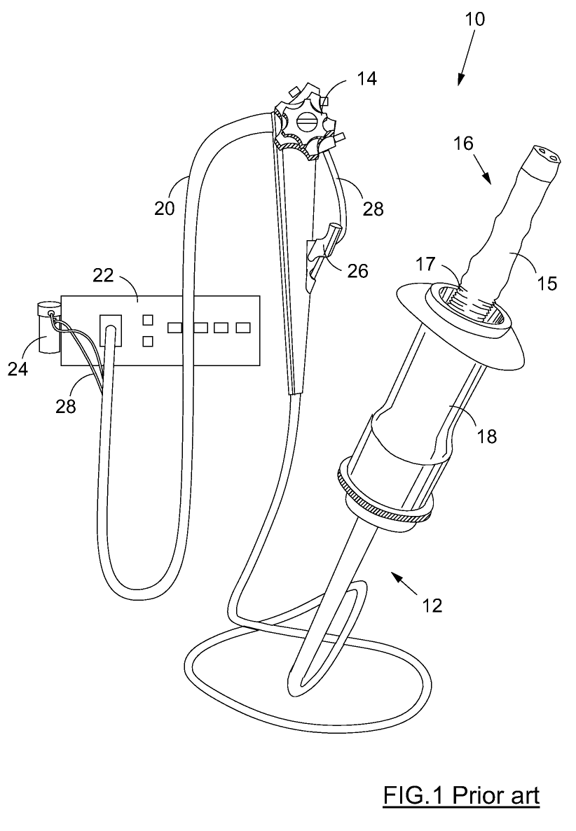

With reference to FIG. 1 there is shown a prior art colonoscopic apparatus, described in Bar-or (International patent application PCT/IL05/000425; Publication WO 2005/110204). This apparatus employs an inflatable propelling sleeve and comprises main components, which can be used also for implementation the present invention, as it will be apparent further. The colonosocopic apparatus 10 comprises an endoscope having an insertion tube with its proximal section 12 connected to an operating handle 14 and with its distal section 16 protruding from a disposable dispenser 18. An example of a suitable dispenser and explanation of its functioning can be found in Eizenfeld (International application PCT/IL03/00661; Publication WO 2004/016299).

It is not seen in FIG. 1, but one should bear in mind that the distal section of the endoscope is provided with a bending section for maneuvering the endoscope during its advancement within a body passage and with a probe, which terminates at its forwardmost end by an appropriate optical head enabling visualization of the colon's interior. It is seen in FIG. 1 that a flexible sleeve covers the protruding distal section of the endoscope. That part of the sleeve, which is seen in FIG. 1, comprises a frontal noninflatable portion 15 and a rear, folded portion 17, which before advancement of the insertion tube is retained being folded within the dispenser. The frontal portion of the sleeve covers the bending section of the endoscope and its head. The frontal portion does not inflate when the endoscope advances within the colon. The rear portion of the sleeve covers the insertion tube and unfolds when air or other fluid medium is supplied to the sleeve. By virtue of this provision, when the sleeve is inflated, the insertion tube is propelled within the colon. Explanation of this phenomenon is given in the above referred-to references.

The endoscopic apparatus of the present invention is of a similar type in the sense that it employs the same main components and the same propelling mechanism, based on inflation of a flexible disposable sleeve coupled to the endoscope's distal section.

It should be appreciated also that the present invention is not limited merely to colonoscopy. It can be employed in any other medical procedure requiring insertion of a probe in a body passage for inspection of its interior or in any technical field, requiring advancement of a long, pliable object through a tube or conduit having a complex shape consisting of turns and loops.

The main idea of the present invention is to provide the endoscope with at least one additional propelling sleeve, which can be inflated independently of the main sleeve shown in FIG. 1. The additional propelling sleeve upon inflation provides an auxiliary propelling force, which assists in advancement of the endoscope through the complicated body passage irrespective of its configuration and material structure.

It is seen also in FIG. 1, that the operation handle is connected by an appropriate umbilical cord 20 to a control unit 22 provided with a source of compressed air for inflating and venting the sleeve. It is not shown specifically but should be appreciated that the control unit comprises appropriate solenoids for activating pinch valves for closing or opening tubes passing through the cord 20 and supplying air, water or vacuum. A flask 24 is provided, which is filled with water to be supplied under pressure into the body passage when irrigation is required.

It is not shown in detail, but one should bear in mind that the insertion tube is fitted with various devices, which are necessary for proper functioning of the endoscope. These devices are known per se. Among such devices one can mention vertebrae and strings, which can be manipulated by the handle and a working channel, or so-called multilumen tubing provided with passages for supplying water for irrigation, air for insufflation or vacuum for suction or with a passage for introducing surgical instruments as might be required during the endoscopic procedure.

The multilumen tubing extends through the insertion tube and through the handle to a connector means 26, which provides fluid communication with the control unit and via extension tubing 28 with the flask.

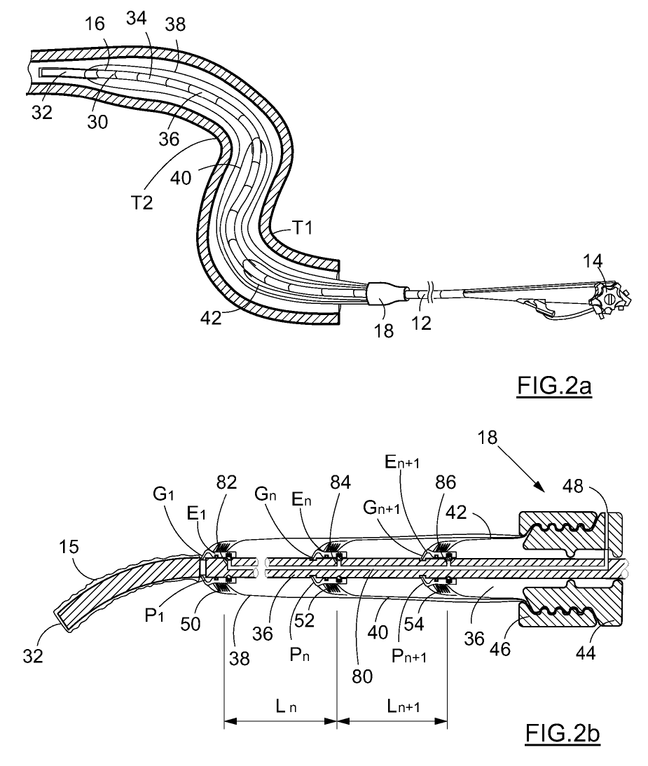

As shown in FIG. 2a, the distal section of the endoscope of the present invention is inserted and advanced within a complicated body passage of a patient, e.g. colon. For the sake of brevity only a small section of the passage is shown, which has Z-like configuration consisting of two sharp turns T1, T2. Disposable dispenser 18 and proximal section 12 of the endoscope remain outside the body passage. A dedicated garment (not shown) can support the rearmost part of the distal section, for example as explained in Golan (International patent application PCT/112004/000372; Publication WO 2004/107889). By virtue of this provision more convenient operation of the endoscope is possible.

It is seen in FIG. 2a that distal section 16 of the endoscope extends along the Z-like section of the body passage and has the same Z-like configuration. The distal section comprises a probe section 30 provided with a camera head 32. It is not shown but should be appreciated that the camera head is fitted with appropriate illumination means and optics as required for visualization the colon's interior. The distal section consists also of a bending section 34, which is located behind the probe section, and of a forward most region 36 of the insertion tube.

In the endoscopic apparatus of the present invention a main flexible sleeve 38 surrounds the major part of the distal section. In addition to the main flexible sleeve there is provided a first auxiliary flexible sleeve 40, which is surrounded by the main sleeve. There is also provided a second auxiliary flexible sleeve 42, which is surrounded by the first auxiliary sleeve. Typically sleeves 38, 40, 42 are manufactured of a flexible, biocompatible plastic, such as polyamide, having a thickness of about 20 microns.

It should be kept in mind that in FIG. 2a is shown the situation when all three sleeves have been already engaged, inflated and fed out from the dispenser. First the main sleeve was inflated to propel the distal section along the body passage until it met an obstacle, e.g. sharp turn T1. Then the first auxiliary sleeve was inflated to provide additional propelling force acting at the distal section and assisting to advance it along the body passage still further, until the distal section meets a new obstacle, e.g. sharp turn T2. At this point the second auxiliary sleeve has been inflated to propel the distal section still further. In accordance with the present invention all sleeves can be selectively engaged and inflated independently one from another. By virtue of this provision the maneuverability of the distal section of the endoscope within the body passage could be significantly improved and advancement of the endoscope through a convoluted body passage becomes much easier.

Referring now to FIG. 2b the endoscope is shown in the situation when all the sleeves are inflated, however for the sake of simplicity the endoscope is shown outside the body passage. It is shown schematically that proximal ends of all sleeves are anchored within dispenser 18. According to the embodiment shown in FIG. 2b the dispenser comprises an inside bushing portion 44 and an outside bushing portion 46. The bushing portions are provided with mating labyrinth sides, which are press fit with proximal ends of the sleeves held therebetween. The bushing portions are press fit or glued into the base of the dispenser (not shown). Anchoring mechanism of the distal end is not shown in detail, however one can use for this purpose the arrangement, which is disclosed in Bar-or (International application PCT/IL05/000425; Publication WO 2005/110204). This arrangement comprises an internal bushing member with a retaining ring and an external skirt member. The sleeve is held folded between the bushing member and the skirt member. An O-ring secures the distal end of the sleeve at the retaining ring. One should appreciate that the above-described anchoring mechanisms are only examples and that anchoring mechanisms having other construction can be used for the same purpose.

It is seen also in FIG. 2b that the forward most, non-inflatable portion 15 of the distal section terminates with the optical head 32. The distal end of each sleeve is connected to the insertion tube by virtue of an attachment mechanism, which causes engagement of the sleeve by the insertion tube when it advances within the dispenser. This attachment mechanism can be identical with or similar to the snapping arrangement disclosed in Bar-or (International application PCT/IL05/000425; Publication WO 2005/110204). The snapping arrangement may comprise elongated transversal grooves on the insertion tube into which tongues, protruding from the inwardly facing periphery of the retaining ring, can snap.

The attachment mechanism of the main sleeve 38 is designated by reference numeral E₁, while the attachment mechanisms of the auxiliary sleeves 40 and 42 are respectively designated by reference numerals Eₙ and Eₙ₊₁. As mentioned above the attachment mechanism may include an transversal annular groove made on the insertion tube, which is adapted to be in snapping relationship with a protrusion made on the bushing member. By virtue of the attachment mechanism the sleeve, which has been initially kept folded within the dispenser, can be engaged by the insertion tube and thus be available for inflating and feeding out from the dispenser as soon as the protrusion is brought into snapping engagement with the groove.

In FIG. 2b is schematically shown that the attachment mechanism of sleeve 38 comprises groove G₁ made on the insertion tube and protrusion P₁, attachment mechanism of sleeve 40 comprises groove Gₙ and protrusion Pₙ and attachment mechanism of sleeve 42 comprises groove Gₙ₊₁ and protrusion Pₙ₊₁.

Particular locations of the grooves on the insertion tube are selected in such a manner that the attachment mechanism is triggered as soon as the distal end of the insertion tube reaches an obstacle within the body passage. The distance between dedicated locations is designated in FIG. 2b as Lₙ and Lₙ₊₁. The amount of dedicated locations, their position along the insertion tube and the distance therebetween is selected empirically and in accordance with the configuration of the body passage, through which the endoscopes should be advanced.

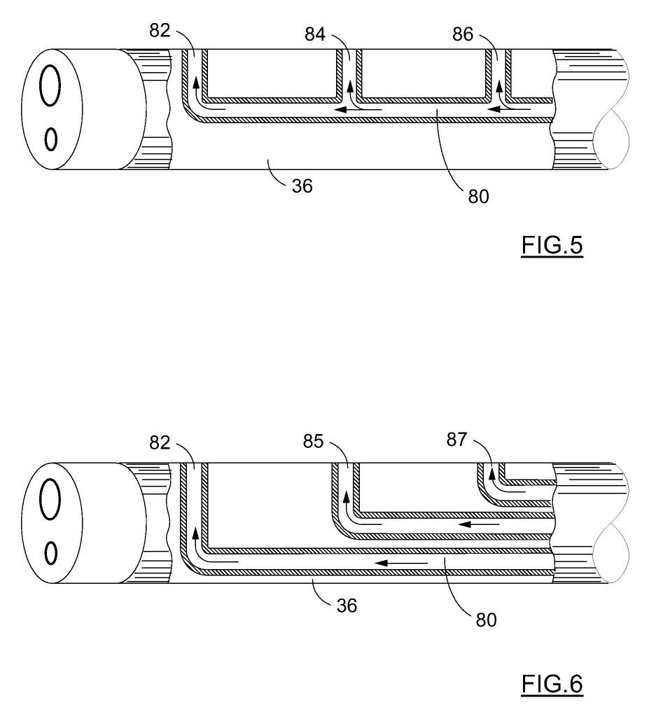

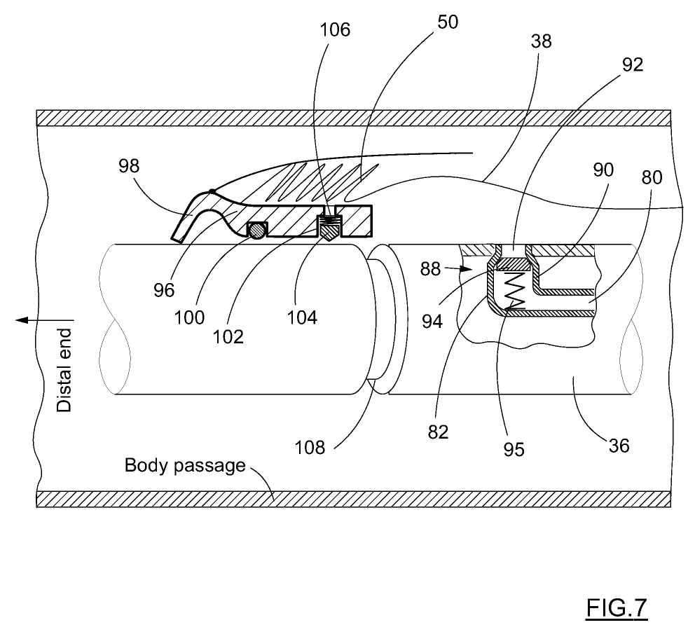

It is also seen in FIG. 2b, that a short transversal channel 48 is provided within inside bushing portion of the dispenser. Through this channel a suitable fluid medium can be supplied under pressure for inflating the sleeves through respective channels 80, 82, 84, and 86. The channels are depicted in FIGS. 5 and 7. Typically the inflating medium is air, however other fluids can be used for inflating as well.

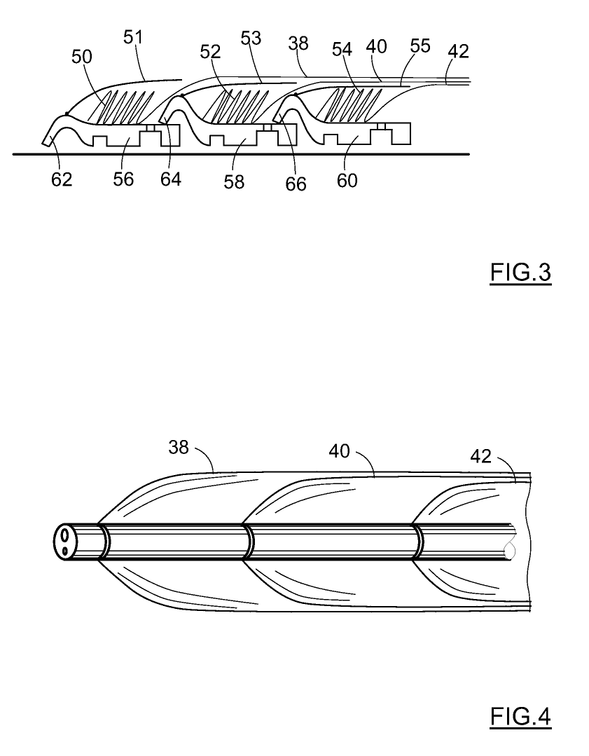

At the beginning of the endoscopic procedure the distal sections of the sleeves are kept folded within the dispenser so that most of their length is bunched. The sleeves become engaged by the attachment mechanism and can be inflated after the distal section of the endoscope has been inserted via the anus and is being advanced through the body passage. As explained above the sleeves are consecutively engageable by their respective attachment mechanisms when the insertion tube advances through the dispenser. FIG. 3 shows a fragment of an embodiment in which sleeves 38, 40, 42 and their bunched portions 50, 52, 54 are held within dispenser 18. The bunched portions are retained between external skirt members 51, 53, 55 respectively associated with each sleeve and respective internal bushing members 56, 58, 60. In accordance with the invention snapping engagement with the insertion tube is ensured by providing each bushing member with a hook-like forward end 62, 64, 66, which is capable of elastically snapping into a respective annular groove provided on the insertion tube. It is seen also that when the sleeves are held within the dispenser the forward hook-like ends 64, 66 of the bushing members rest on rear ends of the adjacent bushing members 56, 58. By virtue of this provision the arrangement is compact and there is no need to increase the dispenser size.

The bunched portions 50, 52, 54 of the sleeves are arranged as a concertina and when the sleeves are engaged and inflating medium is supplied to them the bunched portions begin to unfold and reduce their length on account of unfold and inflated region. This situation has been shown in FIGS. 2a, 2b.

With reference to FIG. 4 is shown the situation in which the endoscope of the present invention is propelled by sleeves 38, 40, 42, which all are engaged by respective attachment mechanisms and inflated.

Attention is now drawn to FIG. 5, schematically depicting how inflating medium can be supplied through the insertion tube to those locations of the insertion tube, where the sleeves are engaged by the attachment mechanism and are available for inflation. Typically the inflating medium is supplied to the insertion tube from a source, which is located in the control unit 22. The inflating medium is supplied along the insertion tube through a main channel 80, which extends longitudinally along the insertion tube 36. In the vicinity of the distal end the main channel terminates by a transversally directed section 82, through which inflating medium can be supplied to the main sleeve. The main channel is provided also with a first and with a second transversal branch 84, 86 through which inflating medium can be supplied to the auxiliary sleeves. According to an alternative embodiment, which is seen in FIG. 6 the inflating medium can be supplied immediately to each sleeve through dedicated channels 85, 87. By virtue of this provision each sleeve can be inflated independently.

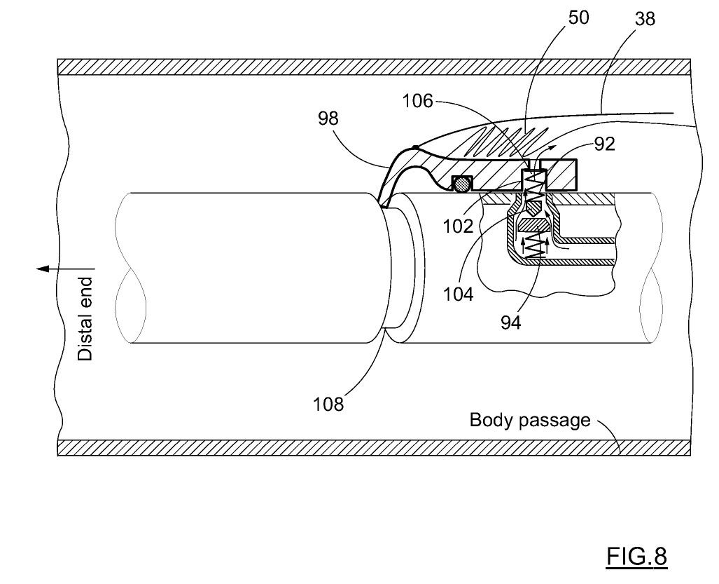

With reference to FIGS. 7, 8 there is shown an embodiment of the attachment mechanism and an embodiment of the automatic, sequential, inflating mechanism for supplying inflating medium to the sleeves. The attachment and inflating mechanism is shown only for one sleeve, which can be either the main sleeve or one of the auxiliary sleeves. It should be appreciated that similar mechanisms are available for the rest of the sleeves.

In FIG. 7 there is a portion of the insertion tube 36 which is advanced along a body passage. Attachment mechanism is seen, which enables engagement of the sleeve 38 by the insertion tube and subsequent inflation of the sleeve. In FIG. 7 is shown that the sleeve is not engaged yet and inflating medium is not supplied to the sleeve. Extending along the insertion tube the main channel 80 is seen. The channel terminates by a transversal branch 82, in which resides a valve means 88. The transversal branch has a narrowing transient portion 90 terminating by an opening 92. The diameter of the transversal branch 82 exceeds the diameter of the main channel and of the opening. The valve means comprises a spring loaded floating closure 94, which has a conical shape mating with the shape of the transient portion. The closure is retained in the floating position by a spring 95. Since the closure is not secured firmly within the channel it is urged by the spring and by pressure of the inflating medium to float up, to seal the opening and thus to prevent the escape of the inflating medium from the main channel.

The attachment mechanism comprises an internal bushing 96, provided with an elastically bendable hook-like forward portion 98. An O-ring 100 is provided to ensure reliable sealing engagement between the bushing and the insertion. In the middle part of the bushing a recess 102 is provided, which communicates with the bunched portion 50 of the sleeve through a short opening. Located within the recess with a possibility for displacement therealong a small plunger 104 is provided, which is loaded by a spring 106. An annular groove 108 is provided on the insertion tube. The arrangement being such that when the hook-like portion 98 is not in snapping engagement with the groove the outwardly facing surface of the insertion tube abuts conical end of the plunger and forces it to hide within the recess 102.

The distance between the center of the opening 92 and the middle plane of the groove is deliberately selected to be equal to the distance between the hook-like portion 98 and the center of the recess 102. By virtue of this provision when the hook-like portion is in snapping engagement with the groove 108, the recess is opposite the opening 92.

This situation is shown in FIG. 8. The spring urges the plunger to push closure 94 down so as to allow the inflating medium to flow through opening 92 and through recess 102 to inflate bunched portion 50 of sleeve and thus to assist the advancement of the insertion tube along the body passage.

Thus by providing the insertion tube with at least one additional auxiliary sleeve, it is possible to create additional propulsion force distributed along intermediate sections of the insertion tube between its distal and proximal end. This additional propulsion force assists to advance the endoscope within a body passage. The additional propulsion force can be applied selectively in those locations of the body passage, where the tube meets such unavoidable obstacles, like sharp turns etc. Furthermore, the additional propulsion force can prevent entanglement of the insertion tube due to stumbling with the obstacle.

Those skilled in the art will readily appreciate that the invention may be implemented with any number of auxiliary sleeves. The herein description showed two auxiliary sleeves for illustrative purposes only and it was not intended to limit the invention to just two auxiliary sleeves. The specific number of sleeves will be determined by the specific configuration of the cavity in which the device is being utilized.

It should be appreciated that the present invention is not limited to the above-described embodiments and that changes and one ordinarily skilled in the art can make modifications without deviation from the scope of the invention, as will be defined in the appended claims.

For example instead of supplying the inflating medium from a common source one could use separate sources, each of them being dedicated to a certain sleeve and connected to it directly. The inflating medium can be supplied to the sleeves either under constant pressure or under variable pressure. The propelling sleeves can be inflated and engaged simultaneously or according to the desired sequence.

It should be readily appreciated that by virtue of this arrangement it is possible to distribute the propelling force along the insertion tube, which would improve the maneuverability of the insertion tube within the conduit.

One can contemplate a design in which the engagement and inflating of the sleeves can be carried out sequentially, i.e. one after another, or simultaneously or in any desired combination.

The present invention can be also implemented not only as an endoscope suitable for investigating of body passages, but also as a borescope for use in various engineering applications, in which it is required to investigate the interior of complicated tubular passages or to pass an object therethrough. One can contemplate that in such an endoscope, instead of an insertion tube, a suitable shaft can be employed.

It should also be appreciated that the features disclosed in the foregoing description, and/or in the following claims, and/or in the accompanying drawings may, both separately and in any combination thereof, be material for realizing the present invention in diverse forms thereof.

When used in the following claims, the meaning of terms “comprise”, “include”, “have” and their conjugates is “including but not limited to”.

I claim:

1. An apparatus for endoscopic examination of a tubular conduit, said apparatus being provided with an insertion tube fitted at a distal end thereof with an optical head for visualization of the conduit's interior during the advancement therethrough, said insertion tube being coupled with a major inflatable sleeve which upon inflation propels the endoscope within the conduit, wherein the improvement comprises at least one auxiliary inflatable sleeve coupled with said insertion tube, wherein upon inflation said at least one auxiliary sleeve provides additional propelling force for advancement of the insertion tube within the conduit.

2. The apparatus as defined in claim 1, wherein said conduit comprises a body channel.

3. The apparatus as defined in claim 2, wherein said body channel comprises a colon.

4. The apparatus as defined in claim 1, wherein said major sleeve and said at least one auxiliary sleeve are made from a flexible, biocompatible plastic.

5. The apparatus as defined in claim 1, further comprising a dispenser retaining therein the major sleeve and the at least one auxiliary sleeve, wherein said major sleeve and said at least one auxiliary sleeve beings retained in the dispenser in a folded state and upon inflation feed out therefrom in an extended state.

6. The apparatus as defined in claim 5, wherein said major sleeve surrounds the at least one auxiliary sleeve.

7. The apparatus as defined in claim 5, wherein said dispenser is provided with an inside bushing portion and with an outside bushing portion, wherein proximal ends of the major sleeve and of the at least one auxiliary sleeve being anchored between the inside bushing portion and the outside bushing portion, and wherein distal ends of the major sleeve and of the at least one auxiliary sleeve being coupled to the insertion tube.

8. The apparatus as defined claim 7, wherein said distal ends of the major sleeve and of at least one auxiliary sleeve being secured at the insertion tube by an attachment mechanism causing engagement of the major sleeve and of at least one auxiliary sleeve upon advancement of the insertion tube through the dispenser.

9. The apparatus as defined in claim 8, wherein said attachment mechanism comprises an annular groove made on the insertion tube and a protrusion made on a bushing member put over the insertion tube, said protrusion being in a snapping relationship with the groove.

10. The apparatus as defined in claim 8, wherein said attachment mechanism comprises an annular groove made on the insertion tube and a hook-like end formed on a bushing member put over the insertion tube, said hook-like end being in a snapping relationship with the groove.

11. The apparatus as defined in claim 8, wherein said major sleeve and said at least one auxiliary sleeve being connected to the insertion tube by separate attachment mechanisms.

12. The apparatus as defined in claim 1, wherein said major sleeve and said at least one auxiliary sleeve being in fluid communication with a source of a fluid medium supplied for inflating the major sleeve and the at least one auxiliary sleeve.

13. The apparatus as defined in claims 12, wherein said major sleeve and the at least one auxiliary sleeve being in fluid communication with a common source of the fluid medium.

14. The apparatus as defined in claim 8, wherein said major sleeve and said at least one auxiliary sleeve being consecutively engageable by respective attachment mechanisms upon advancement of the insertion tube through the dispenser.

15. The apparatus as defined in claim 12, wherein said at least one source is in fluid communication with the major sleeve and with at least one auxiliary sleeve through a channel extending within the insertion tube, said channel having a common, longitudinal section and transversally directed branch sections.

16. The apparatus as defined in claim 10, further comprising an inflating mechanism for supplying the fluid medium to the major sleeve and to the at least one auxiliary sleeve.

17. The apparatus as defined in claim 16, wherein said inflating mechanism comprises a main channel for supplying the fluid medium to the major sleeve and to the at least one auxiliary sleeve, said main channel terminating by a transversal branch fitted with a floating closure, wherein said attachment mechanism comprises a recess made in the bushing member and a spring-loaded plunger residing in the recess, the arrangement being such that when the hook-like end of the bushing member engages the groove, the recess is brought opposite the transversal branch and the plunger urges the floating closure to admit the fluid medium from the main channel to the major sleeve and to at least one auxiliary sleeve.

18. A method for endoscopic examination of a tubular conduit, said method comprises
providing an insertion tube, which is fitted at a distal end thereof with an optical head enabling visualization of the conduit's interior,
coupling of a major sleeve to the insertion tube, said sleeve being inflatable to propel the insertion tube through the conduit,
coupling of at least one auxiliary inflatable sleeve to the insertion tube, said at least one auxiliary sleeve being inflatable to provide additional propelling force for advancement of the insertion tube within the conduit.

19. The method as defined in claim 18, wherein upon inflation of said major sleeve and of said at least one auxiliary sleeve a propulsion force is created, which is distributed along the insertion tube.

20. The method as defined in claim 19, wherein said major sleeve and said at least one auxiliary sleeve are inflated independently.
